(19) United States
(12) Patent Application Publication
(10) Pub. No.: US 2010/0284510 A1
(43) Pub. Date: Nov. 11, 2010

(54) IMAGING GALLBLADDER USING COMPUTED TOMOGRAPHY

(76) Inventors: Jeffrey Neitlich, Miami Beach, FL (US); Tyler Neitlich, Miami Beach, FL (US)

Correspondence Address: K&L Gates LLP, 1900 MAIN STREET, SUITE 600, IRVINE, CA, 92614-7319, US

(21) Appl. No.: 12/463,077

(22) Filed: May 8, 2009

(51) Int. Cl. A61B 6/00

(52) U.S. Cl. 378/6

(57) ABSTRACT

Methods are described for detecting gallbladder complications, e.g. gallstones, in overweight patients using a contrast agent and computed tomography (CT). The methods are generally used prior to bariatric surgery to reduce possible complications associated with the gallbladder following the surgery. The methods described replace existing ultrasound imaging of the gallbladder as the present methods can image the gallbladder through fatty adipose tissue, which ultrasound cannot.

### BACKGROUND

Bariatric surgeries have become one of the most commonly performed elective surgical procedures in the United States, with over 100,000 new cases being performed each year. The number of performed weight loss surgery procedures has increased by more than 600% in the last ten years. Bariatric surgery candidates are overweight and generally have a number of comorbidities that place them at risk for complications. These candidates often exhibit other medical complications aside from simply being overweight, for example, candidates can have co-existing diabetes, hypertension, and/or cardiovascular disease. Additionally, these patients are at high risk for having, or even developing gallstones.

Cholelithiasis, or the formation of gallstones, is more prevalent among overweight people, and hence bariatric surgery candidates. In addition, the risk of cholecystitis, or inflammation of the gallbladder resulting from the presence of gallstones in the gallbladder or cystic duct, is also relatively high in patients after bariatric surgery, as their rapid weight loss can increase the rate of formation of gallstones. After bariatric surgery, cholecystitis is much more difficult to treat, and the morbidity and mortality become much more elevated. Studies have shown that the majority of patients undergoing some form of weight reduction surgery have gallstones or gallbladder disease, approximately one-third of post bariatric surgery patients will develop gallstones, and the majority of these will need intervention, either medical or surgical.

As a result of the high risk of complications associated with the gallbladder, ultrasound evaluation of the gallbladder is part of the routine preoperative workup of bariatric surgery patients. Patients who have gallstones, gallbladder disease or at least one other gallbladder complication, will either undergo cholecystectomy during their bariatric procedure, or will undergo elective cholecystectomy prior to their bariatric procedure to avoid any complications associated with the gallbladder.

### DEFINITION OF TERMS

Certain terms as used in the specification are intended to refer to the following definitions, as detailed below. Where the definition of terms departs from the commonly used meaning of the term, applicant intends to utilize the definitions provided below, unless specifically indicated.

As used herein “controlled release” refers to the release of a contrast agent from a delivery medium at a predetermined rate. Controlled release implies that the contrast agent does not disperse from the delivery medium sporadically in an unpredictable fashion and does not “burst” away from the delivery medium upon contact with a biological environment (also referred to herein as first order kinetics) unless specifically intended to do so. However, the term “controlled release” as used herein does not preclude a “burst phenomenon” associated with administration. In some embodiments an initial burst of contrast agent may be desirable followed by a more gradual release thereafter. The release rate may be steady state (commonly referred to as “timed release” or zero order kinetics), that is the contrast agent is released in even amounts over a predetermined time (with or without an initial burst phase) or may be a gradient release. A gradient release implies that the concentration of contrast agent released from the delivery medium changes over time.

As used herein “overweight” refers to patients who are over clinically acceptable standard for weight based on age and height. For example, overweight can refer to a patient that is at least greater than about 10% above ideal body weight, more preferably greater than about 20%. Ideal body weight is a clinically set weight that is acceptable based on a patient's age and weight. Another mode of defining overweight is a patient displaying a body mass index greater than about 20% above normal, more preferably greater than 10% above normal. Body mass index is a measurement which compares a patient's weight and height and is commonly known in the art.

### DETAILED DESCRIPTION OF EXAMPLE EMBODIMENTS

Limitations exist when using ultrasound to image the gallbladder in obese patients, or even simply overweight patients, as ultrasound is not efficient in penetrating fatty tissue. Since the gallbladder is located in the abdomen, a region where a majority of the fatty tissue in an obese patient is stored, imaging of the gallbladder can sometimes be impossible. False negative rates of up to 20% have been reported in obese patients when looking for gallstones using ultrasound imaging techniques. Clinically, such a high false negative rate is unacceptable, and as a result, more sensitive imaging techniques need to be developed in order to properly evaluate an overweight or obese patient prior to bariatric surgery.

Some example embodiments described in the present disclosure are generally related to the field of medical imaging, more specifically to the detection of gallbladder complications and abnormalities in overweight patients using computed tomography.

Some of the example embodiments described in the present description overcome the shortcomings of present ultrasound imaging of fatty tissue and provide a method of imaging that dramatically aids in the diagnosis of gallbladder conditions for overweight or obese patients.

The present description provides example methods of imaging and detecting abnormalities in the gallbladder and bile duct system, particularly in overweight patients. The example methods generally utilize computed tomography (CT) with the aid of at least one contrast agent to image the region of interest. The example methods described herein are superior to commonly used ultrasound imaging techniques when used on overweight patient populations.

Generally, example methods are described herein for detecting gallstones comprising: (a) selecting a patient; (b) administering an oral contrast agent to the patient; (c) imaging the patient using CT thereby producing imaging results; (d) analyzing the imaging results; and (e) detecting a presence or an absence of gallstones in the patient. In one embodiment, the CT is performed on the abdomen of the patient.

In one embodiment of the example methods, the patient is overweight and a candidate for at least one bariatric surgery. In still another example embodiment, the overweight patient is greater than 20% above ideal body weight. In yet another example embodiment, the at least one bariatric surgery is selected from the group consisting of biliopancreatic diversion, jejuno-ileal bypass, vertical banded gastroplasty, laparoscopic adjustable gastric band, sleeve gastrectomy, transoral gastroplasty, gastric bypass surgery, implantable gastric stimulation, and combinations thereof.

In still another example embodiment, the methods further comprise determining a presence or a large presence of gallstones in the overweight patient based on analysis of the imaging results. If the presence of gallstones is determined, the example methods further comprise conducting a cholecystectomy concurrently with the bariatric surgery. In still other example embodiments, the administration of the contrast agent can be in the form of a tablet, and the tablet can have a controlled release of about 12 hours. In still further example embodiments the oral contrast agent is an iodine containing organic acid with a triiodobenzene core and can be selected from iocetamic acid, iopanoic acid, ipodate, tyropanoate, salts, derivatives and combinations thereof.

Further, example methods are described herein for detecting gallstones in an overweight patient comprising: (a) selecting an overweight patient who is a candidate for a bariatric surgery; (b) administering an oral contrast agent to the overweight patient; (c) imaging the overweight patient using CT thereby producing imaging results; and (d) analyzing the imaging results thereby detecting a presence or an absence of gallstones in the overweight patient.

In one example embodiment, the bariatric surgery is selected from the group consisting of biliopancreatic diversion, jejuno-ileal bypass, vertical banded gastroplasty, laparoscopic adjustable gastric band, sleeve gastrectomy, transoral gastroplasty, gastric bypass surgery, implantable gastric stimulation, and combinations thereof.

In another example embodiment, the overweight patient is greater than 20% above ideal body weight or is greater than 20% above ideal body mass index. In yet another example embodiment, the overweight patient is clinically obese.

In still other example embodiments, the administration of the contrast agent can be in the form of a tablet, and the tablet can have a controlled release of about 12 hours. In still further example embodiments the oral contrast agent is an iodine containing organic acid with a triiodobenzene core and can be selected from iocetamic acid, iopanoic acid, ipodate, tyropanoate, salts, derivatives and combinations thereof.

Further still, described herein are example methods of detecting gallstones comprising: (a) selecting an overweight patient who is a candidate for a bariatric surgery, wherein the overweight patient is greater than 20% above ideal body weight; (b) administering an oral contrast agent selected from the group consisting of iocetamic acid, iopanoic acid, ipodate, tyropanoate and combinations thereof in the form of a controlled release tablet to the overweight patient; (c) imaging the gallbladder of the overweight patient using CT thereby producing imaging results of the gallbladder; (d) analyzing the imaging results thereby determining a presence or an absence of gallstones in the overweight patient; and (e) conducting a cholecystectomy concurrently with the bariatric surgery if the presence of gallstones in the overweight patient is determined.

The present description provides methods of imaging and detecting abnormalities and complications in the gallbladder and bile duct system, particularly in overweight patients. The method generally utilizes at least one contrast agent which has an affinity for the gallbladder and bile ducts, and CT to image the abdominal region.

Complications in the gallbladder include, for example, cholelithiasis, the formation of gallstones, and cholecystitis, an inflammation of the gallbladder resulting from gallstones present in the gallbladder and/or cystic duct. Another common gallbladder complication includes choledocholithiasis which is an inflammation resulting from gallstones present in the common bile duct. Several other complications are known in the art, but for the sake of brevity, will not be listed here.

The example methods described herein begin with the selecting of a patient or group of patients in need of imaging to detect the presence or absence of gallbladder and/or bile duct system abnormalities and who would benefit from the example methods described herein. Such patients include those who are medically or clinically overweight. Overweight as used herein describes a patient that is at least greater than about 10% above ideal body weight, more preferably greater than about 20%. Overweight patients are preferred candidates because the methods described herein provide superior imaging results when compared to traditional ultrasound as the patient's weight increases. In other words, the more overweight a patient is, the more pronounced advantage the example methods described possess over traditional ultrasound techniques in imaging the gallbladder and common bile duct. In a preferred, non-limiting embodiment, the patient is clinically obese.

The example methods described herein work equally well on patient's who are not overweight. However, for such patients, wherein conventional ultrasound imaging signals are not depleted as a result of excessive fatty adipose tissue, conventional ultrasound techniques can be used.

For the overweight population, it is highly common in today's society for individuals to strive to look and feel their best, if not for health, for vanity. There are several procedures available today to trim or even completely remove all indications of being overweight or obese. One of the most common surgical methods to treat obesity is gastric bypass, wherein a major portion of the intestines is bypassed in an effort to avoid unwanted absorption of nutrients from ingested food. Gastric bypass surgeries have proven to be highly successful in treating obesity.

Other common forms of bariatric surgery include biliopancreatic diversion, jejuno-ileal bypass, vertical banded gastroplasty, laparoscopic adjustable gastric band, sleeve gastrectomy, transoral gastroplasty, implantable gastric stimulation, and combinations thereof. The different forms of treatment can be used in combination, but typically are used individually for safety reasons.

One major concern for any type of bariatric or weight reducing procedure is the presence or eventual formation of gallbladder complications, such as cholelithiasis and cholecystitis. As previously stated, the majority of patients undergoing bariatric or weight reducing procedures have gallstones or other gallbladder complications and require treatment. If untreated, patients run the risk of post surgery complications and an increased morbidity resulting from complications from an untreated gallbladder.

The gallbladder is a small non-vital organ located in the abdominal region adjacent to the liver. The gallbladder is connected to the liver and major duodenal papilla and stores bile from the liver in an effort to sequester sufficient bile for bolus food digestion. Release of bile from the gallbladder into the intestines stimulates the secretion of cholecystokinin (CCK), which in turn stimulates the digestion of fats and proteins. The bile also directly emulsifies fats and neutralizes acids in partly digested food.

Any disruption of this system can have adverse effects on a patient. Disruptions include stones formed in the gallbladder, cystic duct and/or common bile duct. Such disruptions can lead to infections within and around the gallbladder. Studies have shown that overweight and obese people are at a much higher risk of developing complications with the gallbladder, especially if gallstones are present before a bariatric surgery.

As a result of the high risk of complications with the gallbladder either during bariatric surgery or after surgery, ultrasound evaluation of the gallbladder has become part of the routine preoperative workup of bariatric surgery patients. Patients who have gallstones, will either undergo cholecystectomy in conjunction with their bariatric procedure, or will undergo elective cholecystectomy prior to their bariatric procedure avoiding any complications associated with the gallbladder.

However, conventional ultrasound techniques are not highly sensitive when patients are overweight. In fact, the more overweight a patient becomes, the more complicated imaging of the gallbladder becomes using ultrasound, to the point of being completely unreliable as an imaging method. This loss in imaging power is due largely to ultrasounds inability to penetrate fatty adipose tissue.

As a result of ultrasounds inability to properly or efficiently diagnose problems with the gallbladder in overweight patients, the present disclosure provides an imaging technique to overcome the shortcomings of ultrasound on overweight patients.

The present example methods utilize CT to image the gallbladder and common bile duct, preferably prior to bariatric surgery avoiding post surgical complications. However, bariatric surgery is not a prerequisite for imaging a gallbladder according to the example methods of the present description. The example methods may also be useful in diagnosing gallbladder complications in overweight patients independent of a bariatric surgery as overweight individuals are at high risk for such complications and may require treatment.

With any form of CT, it is commonly required that the patient be administered a contrast agent that is visualizable using CT. Any method commonly known in the art for administering a contrast agent is within the scope of the present description. For example, intravenous (IV) injection of a contrast agent is a commonly used method of delivery, wherein the contrast agent rapidly distributes through the body and in some cases can sequester at a particular area in the body for imaging. It is not uncommon for a patient to be administered IV contrast agent just prior to imaging because the contrast agents rapidly distribute in the body. Once infused, patients can enhance distribution of the contrast agent by moving around (e.g. roll on the floor) to more rapidly distribute the contrast agent.

Some patients do not react well to contrast agents delivered via IV. It is believed that the rapid distribution of the contrast agent into tissue is the culprit in unpleasant side effects such as, but not limited to nausea, vomiting, diarrhea, and dizziness. In patients who are not suspected to tolerate IV infusion of a contrast agent well, oral, or even rectal doses of contrast agent can be administered.

Oral doses of contrast agent can take the form of tablets, liquids or gels. Liquid contrast agents are well known in the art and commonly flavored for better patent tolerance during consumption. Tablets, on the other hand, can take the form of solid or gel chewable versions or can be a solid or gel non-chewable versions. Oral doses of contrast agents according to the present description tend to distribute into and through the body much slower than an IV dose. It is slow distribution that is believed to reduce unpleasant side effects commonly exhibited with IV administration.

The oral doses can also be formulated as controlled release tablets. For example, the contrast agent can be introduced into a polymeric capsule or into a polymeric matrix capable of controlled release of the contrast agent over about a 12 hr period. The polymeric capsules or polymeric matrices can be formulated to controllably release the contrast agent in a predetermined amount of time, for example about 6 hr to about 12 hr or about 12 hr to about 24 hr.

Solid or gel tablets can also be formulated for rectal administration. Such rectal administered contrast agents can be absorbed by tissues of the rectum and enter the body's circulation. Methods of delivering bioactive agents rectally are commonly known in the art.

Another option for contrast agent delivery is by direct injection to the abdominal region, preferably close to the gallbladder. The direct injection keeps the contrast agent more localized than IV delivery. This localized effect keeps patients from experiencing some, if not all of the unpleasant side effects. Direct injection in a controlled release gel can aid in slower diffusion into the surrounding tissues.

Contrast agents useful according to the present methods include any contrast agent that can be used to image the gallbladder, cystic duct or common bile duct. Particularly useful contrast agents include organic acids containing at least one radiopaque iodine atom and which have an ability to sequester in the gallbladder and common bile duct. Such compounds generally have a triiodobenzene core structure and comprise at least one acidic group and at least one amine functionalized group associated with the aromatic core. Organic iodine compounds when used as contrast agents block x-rays as they pass through the body, thereby allowing body structures containing iodine to be delineated in contrast to those structures that do not contain iodine. The degree of opacity produced by these iodinated organic compounds is directly proportional to their iodine content. Cholecystographic contrast agents are concentrated in the functioning gallbladder and some may provide visualization of the bile ducts.

Contrast agents incorporating heavy iodine atoms include iopanoic acid, its derivatives, salts, analogues and combinations thereof. Iopanoic acid has the following chemical structure:

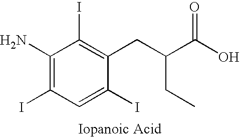

Iopanoic acid appears as a vivid white color (dense) under x-ray radiation, mainly due to the presence of the heavy iodine atoms. Derivatives of iopanoic acid such as iocetamic acid having a chemical structure

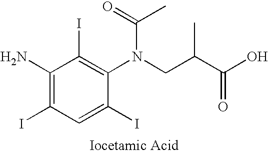

are also useful as a contrast agent according to the present description. Iocetamic acid has a similar core structure to iopanoic acid with three heavy iodine atoms. Iocetamic acid also appears vivid white under x-ray radiation. A further derivative of iopanoic acid is ipodate having a chemical structure

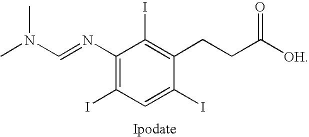

Ipodate also has three heavy iodine atoms and will appear vivid white under x-ray radiation. Further still, another derivative of iopanoic acid is tyropanoic acid having the chemical structure

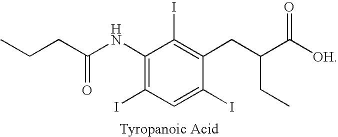

Again, tyropanoic acid has three similar heavy iodine atoms and will appear vivid white under x-ray radiation.

Further, contrast agent formulations useful for imaging the gallbladder and common bile ducts includes those having tradenames BILIVIST® (Berlex Laboratories, Inc., DE) which is an oral form of ipodate, BILOPAQUE® (NY COMED, Inc, DE) which is an oral form of tryropanoate, CHOLOGRAFIN® (Bracco Diagnostics, Inc, NJ) which is an IV form of an amine derivative of iopanoic acid, CHOLEBRINE® (Dagra Pharma B.V., Netherlands) which is an oral form of iocetamic acid, ORAGRAFIN® (Olin Mathieson Chemical Corp., VA) Calcium and Sodium which are an oral forms of ipodate, and TELEPAQUE® (Sterling Winthrop Inc., NY) which is an oral form of iopanoic acid

Once a patient population has been selected, a contrast agent has been administered and an appropriate amount of time has lapsed allowing the contrast agent to distribute itself through the body and sequester in and around the gallbladder, the patients' abdomen region is imaged. Any form of CT can be used to image the abdomen region.

Most CT machines have a weight limit on their examination tables, that weight limit typically being about 550 lbs. Any conventional CT machine can be used according to the example methods described herein without modification. However, if a patient weighs more than the allowable weight limit of the table, modification to the examination table may need to be remedied, as the functioning of the CT machine itself will not change nor will the example method. The example method may also work on patients of any weight barring modification of the examination table.

The methods described herein generally need a CT scan run from at least about the bottom of the lungs to at least about the top of the pelvis of a patient. This is a general guideline because depending on the fatty tissue distribution of the patient, severity of the gallbladder abnormality and the like, a larger area may be scanned. Alternatively, a smaller area can be scanned if the particular circumstances allow.

The resulting CT scan results allow a physician, or even a technician, to easily and properly diagnose an abnormal condition of the gallbladder. Imaging the gallbladder through fatty adipose tissue is not an issue for CT, so visualization of abnormalities or complications such as, but not limited to, gallstones is superior when compared to conventionally used ultrasound methods. Clear two-dimensional and three-dimensional CT images can be properly assessed for any abnormalities of the gallbladder, cystic duct and even the common bile ducts.

The resulting CT scan results are superior to ultrasound when used on overweight patients because of ultrasound's difficulty in imaging a tissue structure through fatty tissue. Ultrasound, when used on overweight patients has a reported false negative rate of up to 20%, which in most clinical settings is unacceptable. In contrast, the false positive rate of the present methods for use in overweight patients is less than about 10%, more preferably less than about 5%. It is even within the scope of the present description that the false positive rate is less than about 1%.

Additionally, the resulting CT scan results are superior to magnetic resonance imaging (MRI) when used on overweight patients. MRI is plagued with artifacts when used to image the abdominal region of an overweight patient. These artifacts pose problems for proper diagnosis, and therefore, MRI is not commonly used to image the gallbladder in overweight patients. The present methods do not suffer from these problems, and therefore, are superior to MRI when imaging the gallbladder, cystic duct and common bile duct in overweight patients.

### Example 1

### Gallbladder Imaging Using CT and IV Contrast Agents Including an Ultrasound Comparison

Nineteen overweight patients underwent ultrasound examination as part of their preoperative work-up prior to gastric bypass surgery. Two of the 19 patients exceeded the CT table weight limit of 550 pounds, and one reported a contrast allergy. This left a cohort of 16 patients, ranging from age 19 through 46, with a weight range of 267 to 494 lbs.

All patients were kept Non Per Os (NPO, Latin: Nothing by Mouth) for four hours prior to their exam. Upon arrival, the patients were IV infused with 20 mg of CHOLOGRAFIN® (iodipamide meglumine) dissolved in 50 cc of saline. The infusion ran in over a 30 min period. After being administered the contrast agent, patients were scanned on a Phillips IEU-22 ultrasound machine, using a curved 5 MHz transducer (Philips Medical Systems, N.A., Bothell, Wash.). When necessary, scans were also performed with a 2 MHz phased array transducer. The presence or absence of gallstones were recorded by the technologist performing the scan, and then directly reviewed by an attending radiologist. When the technologist could not visualize the entire gallbladder, the attending radiologist rescanned the patient to see if they could visualize the gallbladder in its entirety.

Patients remained NPO until the CT scan was completed. Four hours after the initial injection of contrast agent, patients were scanned on a Siemens volume zoom 4 slice CT scanner (Siemens Corporation, New York, N.Y.). Whenever possible, patients were encouraged to walk around, and roll from side to side prior to the scan, in order to mix the contrast and bile in the gallbladder. Scans were performed from above the gallbladder fossa through the pancreatic head, with 3 mm collimation and 2 mm reconstruction interval. All data was sent to an AQUARIUS® (TerraRecon, Calif.) workstation, for both 2D and 3D image review. All scans were reviewed by two CT body imaging radiologists blinded to the ultrasound results, and the diagnosis was made by consensus.

Both the CT and the ultrasound results were scored as follows:

1=No gallstones
2=probable gallstones
3=definite gallstones
4=indeterminate.

An indeterminate ultrasound was reported when the gallbladder could not be visualized in its entirety. When this occurred, the radiologist rescanned the patient to see if they could visualize the gallbladder in its entirety. An indeterminate CT was reported when the gallbladder did not fully opacity with contrast.

Statistical analysis was performed comparing the results of the two studies. Patients were followed clinically for six months after surgery. Gallstones were confirmed or refuted if the patient underwent cholecystectomy, based on the pathology report. Gallstones were considered absent if a patient had no complications related to gallstones in the six month post operative period, and no positive imaging tests for cholelithiasis. Differences in sensitivity between ultrasound and CT Cholecystography in the diagnosis of gallstones were compared by using the exact form of the McNemar test. For statistical analysis, a score of 1 or 2 was considered positive, and a score of 3 was considered negative.

All three patients with gallstones seen sonographically, under ultrasound, had gallstones on CT. The patient with possible gallstones detected sonographically had definite stones on CT. One patient with an indeterminate ultrasound had definite gallstones on CT. The other indeterminate ultrasound also had an indeterminate CT. One patient with no gallstones sonographically had definite gallstones on CT. No patients with a negative CT had a positive ultrasound. Results of the scans are shown in Table 1.

TABLE 1

| Gallstones | Ultrasound | CT |
|---|---|---|
| Definite | 3 | 6 |
| Probable | 1 | 0 |
| None | 10 | 9 |
| Indeterminate | 2 | 1 |

All six patients having gallstones as evidenced by CT (three of whom also had gallstones on ultrasound) underwent cholecystectomy, two during their bariatric surgery procedure, and four prior to bariatric surgery. The remaining ten patients had no gallstones seen on follow up imaging, and no complications due to gallstones in the six month postoperative period.

Based on this data, CT had about 100% sensitivity and a specificity of about 100%, while ultrasound had about 50% sensitivity. CT had no false negatives, while ultrasound had three. The positive predictive value for CT was 100%. The specificity of CT for gallstones was about 91%.

### Example 2

### Gallbladder Imaging Using CT and an Oral Contrast Agent

A 45 year old 435 lb female patient is scheduled to undergo a gastric bypass surgery. As part of the preoperative examination, she is examined for the presence of gallbladder complications. She is given a tablet for oral ingestion the night before she is to be evaluated for gallbladder abnormalities. The tablet contains iopoanic acid which is provided as a 12 hour controlled release formulation. The patient is instructed to take the tablet before she goes to bed the night before her examination and arrive NPO.

The patient arrives for her gallbladder examination. The contrast agent has had at least 12 hours to circulate in the body and sequester in the gallbladder. The patient's gallbladder and common bile ducts are examined using a Siemens volume zoom 4 slice CT scanner (Siemens Corporation, New York, N.Y.). A scan is performed from above the gallbladder through the pancreatic head, with 3 mm collimation and 2 mm reconstruction interval. All data is sent to an AQUARIUS® workstation, for both 2D and 3D image review. All scans are reviewed by two CT body imaging radiologists and the diagnosis was made by consensus.

The patient is diagnosed as having gallstones and is scheduled to have a cholecystectomy in conjunction with her gastric bypass surgery. This procedure allows the patient to recover completely from the gastric bypass procedure without the complications of cholecystitis, which if unchecked increase her risk of morbidity.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that at least one member of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain example embodiments of this invention are described herein, including the best mode known to the inventors for carrying out the invention. Of course, variations on these described example embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the invention so claimed are inherently or expressly described and enabled herein.

In closing, it is to be understood that the example embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of detecting gallstones, comprising:
(a) selecting a patient;
(b) administering an oral contrast agent to said patient;
(c) imaging said patient using computed tomography (CT) thereby producing imaging results;
(d) analyzing said imaging results; and
(e) detecting a presence or an absence of gallstones in said patient.

2. The method according to claim 1 wherein said patient is a candidate for at least one bariatric surgery selected from the group consisting of biliopancreatic diversion, jejuno-ileal bypass, vertical banded gastroplasty, laparoscopic adjustable gastric band, sleeve gastrectomy, transoral gastroplasty, gastric bypass surgery, implantable gastric stimulation, and combinations thereof.

3. The method according to claim 1 wherein said patient is greater than 20% above ideal body weight.

4. The method according to claim 1 further comprising determining a moderate presence of gallstones in said patient based on analysis of said imaging results.

5. The method according to claim 1 further comprising determining a large presence of gallstones in said patient based on analysis of said imaging results.

6. The method according to claim 1 further comprising conducting a cholecystectomy concurrently with said bariatric surgery.

7. The method according to claim 1 wherein said oral contrast agent is an iodine containing organic acid with a triiodobenzene core.

8. The method according to claim 7 wherein said an iodine containing organic acid with a triiodobenzene core is selected from the group consisting of iocetamic acid, iopanoic acid, ipodate, tyropanoate, salts, derivatives and combinations thereof.

9. The method according to claim 1 wherein said oral contrast agent is in the form of a tablet.

10. The method according to claim 9 wherein said tablet has a controlled release over about 12 hours.

11. A method of detecting gallstones in an overweight patient, comprising:
(a) selecting an overweight patient who is a candidate for bariatric surgery;
(b) administering an oral contrast agent to said overweight patient;
(c) imaging said overweight patient using computed tomography (CT) thereby producing imaging results; and
(d) analyzing said imaging results thereby detecting a presence or an absence of gallstones in said overweight patient.

12. The method according to claim 11 wherein said bariatric surgery is selected from the group consisting of biliopancreatic diversion, jejuno-ileal bypass, vertical banded gastroplasty, laparoscopic adjustable gastric band, sleeve gastrectomy, transoral gastroplasty, gastric bypass surgery, implantable gastric stimulation, and combinations thereof.

13. The method according to claim 11 wherein said overweight patient is greater than 20% above ideal body weight.

14. The method according to claim 11 wherein said overweight patient is greater than 20% above ideal body mass index.

15. The method according to claim 11 wherein said overweight patient is clinically obese.

16. The method according to claim 11 further comprising determining a presence of gallstones in said overweight patient based on analysis of said imaging results.

17. The method according to claim 11 further comprising determining a large presence of gallstones in said overweight patient based on analysis of said imaging results.

18. The method according to claim 11 further comprising conducting a cholecystectomy concurrently with said bariatric surgery.

19. The method according to claim 11 wherein said oral contrast agent is an iodine containing organic acid with a triiodobenzene core.

20. The method according to claim 19 wherein said an iodine containing organic acid with a triiodobenzene core is selected from the group consisting of iocetamic acid, iopanoic acid, ipodate, tyropanoate, salts, derivatives and combinations thereof.

21. The method according to claim 11 wherein said oral contrast agent is in the form of a tablet.

22. The method according to claim 21 wherein said tablet has a controlled release over about 12 hours.

23. A method of detecting gallstones, comprising:
(a) selecting an overweight patient electing to have a bariatric surgery, wherein said overweight patient is greater than 20% above ideal body weight;
(b) administering an oral contrast agent selected from the group consisting of iocetamic acid, iopanoic acid, ipodate, tyropanoate and combinations thereof in the form of a controlled release tablet to said overweight patient;
(c) imaging the gallbladder of said overweight patient using computed tomography (CT) thereby producing imaging results of said gallbladder;
(d) analyzing said imaging results thereby determining a presence or an absence of gallstones in said overweight patient; and
(e) conducting a cholecystectomy concurrently with said bariatric surgery if said presence of said gallstones in said overweight patient is determined.
